SEQUENCE LISTING (23 sequences)

SEQ ID NO: 1 - DNA, 601 bp, Homo sapiens
tgaataggcaactatatgtctaacagttttaagttaaattttttaaaagtagcacatagttataatggtcattttacatattttgccaaataattatcctcacttgaaagaggttattaattcttggcttacaaagggaaaactgttttggttttcaagcaccaaagtttaatacatccagttatctcaagggaatgagggatctgttcactggcaatatcttttttgttttccttttctttttgtgattgaaagtaaggataagggaactgatgtagagacaaggacatgctgaagtacmgaggaggtattggaaaatttctttgccatagccaaccaaattttggaataaatggtactaaaatatctggatgatttgtagtaaaaaaaacattgcttgcttgtctgtctacttgattaataccccttgcctttgtcttttggcttataatcctacatttggaataaataagcctctttccatattttattcctttctgccttcaggaaagtacatcttccctgttttggaaagaaacaaactaactcctggacttctccccagcccttcgtttgagcctgtttccctttccaaggactt

SEQ ID NO: 2 - DNA, 601 bp, Homo sapiens
tgaattcaccctgaatcttttaaataatgaactagagctgtataagagggagggatttacaggaaaattgcattcctacggctactgcttcataggtaatatatgggcaaagttagtaagaatttctaattattaattgtgactcaatggcttagaatatcaaggccgcaattacaaacagtgactaccaaaagagttccttttttcctgcaaacaaaaactgctactgaggtaaaagctagaacacagacccacataatgaagaagagctaaagatgagcttaacgtcatgggtaaatgwaaagcatcacaactgtggaaatggaaatgtgatattttgtgaggggaagaaaatgcttgttttagaactctgaaacagtattactttcattataagtgatgtttctatattttgcagtgatatacttatgtaactaggcatggtatctcttactgtccacagcctgtttgcagggagaagccatttgaaattggtgaattgcagggagaagccatttggaagtggtgaatattgaggatatttttgtgggggagcaggttattttcttaatattagagatgtgggtatggaagacaaggt

SEQ ID NO: 3 - DNA, 501 bp, Homo sapiens
cagcccggcacctagtagacactcagtaaatatttgaatgaatgaatgaggccctttggcagccgggaagcccgtcacagccgttgtctccaccctctcccgggctacaccaggccacgccggtaaagttgtttctcctctaggagaaacattcgctaggccttgttgccaacccgcaggcggctgggcgcttcatcccaccctcacccctttccagccaaggtggctgatgggagtcaggctctcgaggkcgcattgccacgaaacagcgtgtgtgagcgcgttgtccccggcccccggcgccacttcccctcggcctagcagcctggactggggaaggacgggtctgctgtacccgggaggtggaaggaaaagccgaaagcggagaagtgtgcgggaggggagtctccgcgcggaggcagcccggcctcctccagtgcaggctgcgcgctggggagccagccagggtgagtcactcccggccgcttccctggagggcgg

SEQ ID NO: 4 - DNA, 801 bp, Homo sapiens
gtttttcgtttctctgcaaagtcacgttatgtgtaaagccaacaaaaaaatgtttcttaaaggatccaagacgaacaacaaaacaccccaaaattactctcccagatcaacattaaaagtttagcactgacattttcagaagagtaagcaacaatgagccctgcctctcttcacacgcttccaaccttccctacgcacgcactcccagctccagacaccttcgcctttactgcagcgataaaggacagcacacccaccgtcgcgggaggaggaggaagagcctctttttctgcagaagagaaacagttcacatcttgttcctgttgcctgcttctgaaacctaaaaacagaagcagcagttaactactaccacataaatccaggggaaacaaatgcttttyctaggctgtttaagtgtctagaaaattttaaaaaggaataaggaggcgggagcgagggaattataagacacaaatggaagcaaatatcaccagcaagactcgttctaggtgtcgtcttaaagaggaagatttttctggctcagaagtttgctgccactagcagggggcaggcgggaccaaaaggtaggaagggggtggagagacagctgctgccccccacccagaagccaggcagagataacacccaccggctacacagatcgggcgaccagagcgcgccctcgagcccgcaggggcggctcctacccggcgggtcaaggtctgcgaagccccgggacgggcacggctctgaacgacctccaggctgggccctcgggggccctccagtgtgggctcagct

SEQ ID NO: 5 - DNA, 713 bp, Homo sapiens
gttttgagttggttcatggtttcaatattagatacctacacccgtttctacaatgtcatgtctttgtaaagatgtattttcagaggttgctccgataaaaagcaactactacatgaaaatgaatgtagaacaggaaatgacagtgtggtattgtccaatttgattctaaggtttgataagttctacagtgctctacactaagttgtaaggacataactactaagtaagttgttcaaacctatttcttttggcctgggggtgtcgtgaaaaatttgctaagactcagagctccctgaatgggtttggaaacctctgccttagcttttagtttgcttttctgataaaattattgatataaagctagtaccagctgatcatggcttaatatatttcctctcctatgtaatatgtttataacttctgcccagttctttcattttaaaactaatcctattgctaacttgttgtactagtcacttcctggtttttcctgtactggtcttctgcttctayttctgttcatctccttaaactccgaatgccccaagtgaaagttaccattttctaccccttaattttcttctaatcatgtaggctttaaattaggcatcatctttaaactacttctttcatttacctcttattcccattcaaaatgtccatatttcctttctttctcatatctctgctttccctcctttactgtgtgaatc

SEQ ID NO: 6 - DNA, 501 bp, Homo sapiens
atgatccattattatcctgtcaataatcataactttattctccattattcagttaaaattattaaatttcatttatattatccctacaccaaaaataatcaaaacacaaaaacacaaggcttcctgagaattaaaatgaacgaaatctattcatctcttttgctacccctacaattctaggcctaccagcagtagtattaatcattctgtttcccactgtttccaacctctagtcatcaaattagtaaccrattattttctattcaacgatgattaatccaacctgcactaaaacagataataattacccataacattaaaggacaaacctgatcccttataataatatccctaattatcttcactgcctcaaccaacctccttgggcttctaccccattcatttacaccaactatccaattatcaataaacctgggtatagcaatccccctgtgagcaggcgcagtaattacagtcttctgctttaaaacgaaaaactcc

SEQ ID NO: 7 - DNA, 601 bp, Homo sapiens
tggcattaattgttctctaatttgggtaggtttgaaatttttcataataaatattaaaaatactttttgaggaaatgtcgtcatgataaacattagaactgcgttcatctaattacttttagggtatgagtgaaactaaaaagtaagactcatctaaaaataatcatctgttttggccatatccccagtgatacatgaatttaattgtttttattgttttcagaatgaaaaattaaagaggttaaataaaattagctggcaatcaaaaaatattagttatatgaagattcagagatttggktatacttaatgaatacaagctaataaatatttattaagactctactgtgctccaaatactatacagggaggtagggatatgaagataggcttagcatcagaaaaggactgtcccttagtagaaaggctgaactaaaaggaaaagtttgacgtgataggcaaattcaactgacaggtccactacccatagtcccttcaagagctcataattaggaaaagtcatatgtataatatggcactttcaataattaagtgcaataagtgtaagtgcaataattaaggtactgggaaaacataacag

SEQ ID NO: 8 - DNA, 601 bp, Homo sapiens
gggctggaaagtgggataaggactaaggacttctgtctctggattcagcatgctgattatggggaaaagcagacatcaattcctcatgtcttgtaagcatcactgtcctccaaagttggagcccagctcagacagctgcatagtattttatagagaaggtgtgttgttgaaatgttgattgtgcaaattgaattgtgttttaagactactgggaaaaccatttaaaggaatcctattgtaagtcattttgaaaaatggagaattacaccacctccccaaggattttctgttttggtaaatwtcatgtggcaagattacttgaagaagggttccagtgttttgctagcttataatggttactgcataaggaaggtttactcgccagaggtccgcacacattggagaactgggggaatatgtaatacttgtgcagaaatgttttgattcatgtactcagaagatgggatctggatgacacctccagcctattgattcccaagtaaggtgtgatgacaccaccaccagcgaggtgtgctatgaggaatttttgttgatgatgaaaagtgaatgctaactgattgcacaccacgtgccccatacc

SEQ ID NO: 9 - DNA, 610 bp, Homo sapiens
agtgcatggcaagcattccacacaaaaagcatttgcacacacaaagaacttacaatgaaaattatgtcttacaagttaacaagccatgagaatgttctaatcatcaagcattaaaaatacccaaatgcatattcatcccatgcrttcttcgacaagtatttgtgaagtgaccatgctttatcaggcaccgtgctaggtgctagactccagagacacgaagacgaataataactggtcttcgtcaacaagaactcaattgtgagacaaaagccaacttgaggaaaattaagcctatttcaaacatgccttgatctgtgtaacattcataatacataatatcttctttgatctccacagttttctctctacctccagatcagtgcaatctctggttttccacttttttttttctttccaaatgaaaatagagcacaactgtcattttcaatctactaagtcataaaccacaaggcaggctgcaaggaccaaatgctccgttttagaacgagcccagtgcagagaacagagtccacaccacaaggcagggagctgtggtccagatgagtgacatgcccttagtgacctgggtcctggcctccgacccagagac

SEQ ID NO: 10 - DNA, 601 bp, Homo sapiens
cacaaatacttgtcgaagaacgcatgggatgaatatgcatttgggtatttttaatgcttgatgattagaacattctcatggcttgttaacttgtaagacataattttcattgtaagttctttgtgtgtgcaaatgctttttgcgtggaatgcttgccatgcactgaaaaatgctttgtgatagcagttttgaaaaattgagtcattttgccctgattgaagctgggtgaccttgtgcaagtcactttaaccctctgggacttgtggccctcatctgtaaaatgggtagaaggtcagagtgsacctcgtggcctgagaggctagtaccccatgctgcatgtaagtcttacatcctcctctttggtgattggatactgaccctctccccaggggttcctgcacagagctggaattctctctggtgtctttgcattccagtgggtggaatgccttgtttctcagtagcaatgagaaatgttaatgtgaggttatgaaagcaagccaagagccatggaataattaaaacacacacgcacacaaagaaggaaaagcgagagggggaaacacacttaatggacaagggatgagtaaaacccaatgga

SEQ ID NO: 11 - DNA, 610 bp, Homo sapiens
tacagaagtccagagatgagagacatgctcttctgattactcctacccaccctttggtcttggctcctatgtcttttccttgggggaaccttcccagaccaccaccccttgctcaaatggaatcaggctccctggacttctcatgcatgactcctagcatttgcagtgttacccttgtgagtgcttcctctttgctcactactatatccagagcccagcacagcataagttgccagtaaaatgtgctgaatgaacaaatgagttaatgccactgaatctgtatcagccttgccatgactccaggagggtggttaaccagacattatcttccttgctttactgatgagggaatcacggcccagagggcatgtctctcacctgggcttggggcactgcgagaggacccaagygccccaaagggggccatgcctttttcttgttggattctttggcaaccagcacggctctgagcacacagcagatgtggattcaaagtagatggaaatagcaactaaatgacctcagagatggagccaaaacaacttttctgtttgcttggcatcacatttccatcaaagcagactgtggagggaggccattctgtttgcat

SEQ ID NO: 12 - DNA, 694 bp, Homo sapiens
tggatttttctagtaacttcaaggggtaaagactttgaattttttttttggagacagagtctcgctttgtcacccaagctggagtgcagtggtgcgatcacagctcactgcagccttgacctcccaggctcaagcaatcctcctgcctcagcccctcaggtagctgggattataggcacacgctaccacgcctggctattttttgtatattttgtggaggtggggtttcaccgtgttgcccaggctggtctcaaactcctgagctcaagcgatctgcccaccttggcctcccaaagtgctgggattaaaggcgtgagccactgtccctggccaagactctgggtttagtaagcctcttgcaactcactaggagtggatttctaggacctccccactttcttgcttgttgtttcttttgctagccgtaagtccctggagtacactgggtactttatgggaaaaagaggaacagtgtgggacagactttgccctgrgctgcttaacgtgatgtggggaacagaggtgataagagcctgtggcagctcaggcagccctgccatgacctggagttggggaggggtatcaaggataacaagtcttatacacatgccatgctgtttctttggatcaatagatggataatttttaaaaccccctgtatgtatcaaactcactttggagatgtaaggcatgg

SEQ ID NO: 13 - DNA, 701 bp, Homo sapiens
ttgaagagagagcttgctcttggggcagtttgtactgggcgtttagggttataaaaacctagatttgctgaaaacggctctcttttatcttctgacaagaacatttgtgaagaaaacaaaacagaccttcagccatttagtctatacaccgagagaatggtttctacagtatgcctgatttctctgggtctttctaacacrtccatgtatgtgcttgtcagcttctagtgcctccatttcttgatatgaggaaattttgcttaaaaaaaaatctctactgataggacctctgcatattcatgcatgtttttcctccagaactttgtgtcccaccactgtgctaggcagtttacaaatgccatctgatttcattcctgccactacagtctaaggctatattgtccgatacctatccaccaccctcatgtggctatttaaatgttgggtcattaaaattaaatacaattacaaatgctgtccctcagtcacaccagccgcatttcaagtgcacgggtaggctagtggccatcagatcagctcatatagagcagcacgttttcatcaccaccaaaaattctattgggtagcactggtctaaggtccggacatatgtccccattttgtagatgaggaagctgagtcctacagaggttaagttgaccacttagaagaatgaacagatgaaaccttttcattttaca

SEQ ID NO: 14 - DNA, 601 bp, Homo sapiens
aggaaaccaagagaaaccagccaccacccacagctgacagggtctggggggaacagcaaaagagctgacccgcacagtacccccaggctaactgaagagtccctgaagcctacagatcaatttctcttttctaagaaggaaaggggagtataaccttcagtttcctcatgtgtaaaatgaaaaggtttcatctgttcattcttctaagtggtcaacttaacctctgtaggactcagcttcctcatctacaaaatggggacatatgtccggaccttagaccagtgctacccaatagaatttytggtggtgatgaaaacgtgctgctctatatgagctgatctgatggccactagcctacccgtgcacttgaaatgcggctggtgtgactgagggacagcatttgtaattgtatttaattttaatgacccaacatttaaatagccacatgagggtggtggataggtatcggacaatatagccttagactgaagtggcaggaatgaaatcagatggcatttgtaaactgcctagcacagtggtgggacacaaagttctggaggaaaaacatgcatgaatatgcagaggtcctatcagtagagat

SEQ ID NO: 15 - DNA, 748 bp, Homo sapiens
aaatcgaagattaatggctcaatctaggaaacatttaagattctatgtgcaggaagaacagggctgggcaccccttggtatatgtaaatgtgtcctcagttttgctttataggatctctttccacaactagttttaccatgtctacttcagcctcattaaggagttaacactgaccagttgcttcctagaagcaagttttatggtaagcactctatatttaaatactttaatcctgtaggacagggcctcatcttccaaaagatgacaatgaagctcagagaggttaggagtattccacaaggtcacccagcaggcaggggagcagagctgaactctgaactctaaacattaaagcccagcttcttcccactgtgtcaagctgttgttctgcgtgccccagccgccttgcagggagacacggtggaaacaacaggagactggaaattgggacttgcattggaaagctggctcctctccttaccacctgggccacctcaggycaagtgcctcatctccgggggcccatttcctcattggtataaaggggccatgtgggaagtggttcctgtagagattgcacatgtaggcccttcataaaccggtaagcgccacactgacatacagtgtcacccataacctttcattcacgaaccagaaaatcctgcccaatgatgttgccgaacatccactcctgaattttaacgttattactggaacaagcttgtgcaaggtcaggacactgtaaga

SEQ ID NO: 16 - DNA, 501 bp, Homo sapiens
ttgtttggtaacaatggaggggaacgttaaacaagagttgacgacgggaagtgaatcactgaattctcaaacaggctccgtggaaggagcaaagaactgtgggaggggcccctggcagggcctggcggggtgcaggggaatctttggaggctgctctcttctcaccccgccaaaggacaccctttcaactctgcctggctgtaaccaggtagcagcttcatatgtcaggccccatctagctcccaaatcamccccgctacttccgagtttccctggtgttctctgctccttggattaagcaggagggagaaatggagaaagagccttttttctacaaacactgacccaactttgtccccatacaccttaaagtctgaccctgaagaggcagctacaggctctaacagaaggaaaaggctcattcttctctccccgctcaggcatcagctacttaaatggagatgtcctcaaaggaaaatataaaacacagagacaatgtct

SEQ ID NO: 17 - DNA, 636 bp, Homo sapiens
tcctttctgtctaagggtacagacctcctattgtacctaaaaaaaaaaaggttctggttcattggggcaattcacctaagacagcaaagaacccctatgggcaggaaaacaggaattccacacaagcaagaaaggaggacttcagagaagccaggcccacacttaacaatgtcasggtggcaacagccctcctcgattaagccggcctcatcaactaagtggcccttgtctgaaagctgcatccctaaaacagctacaatcaggaacaagcaaagggttccaggtgaggcgtcctcccgctgggtcctagatacttacaactctccccggtatcagcccacccagtcccagctgagattcagagcacctgcccacccaccacctcacccccacttcctagacaggcctaatttacaggcatagaaactcaaaatgctgaacaggcccaagtagtggaggtaaaatgttgcagccacagtatctgaatgtgaggctcctgcactcttggccaccagcatagtgcgctgaggtgcaggtggggacagagcccttggaggtccaattatcttccagctcaaacagtaacccactctgtggctctctgctaagaatgacctctctccatcctcccctcct

SEQ ID NO: 18 - DNA, 601 bp, Homo sapiens
tatacagggcacccattctttgttaccatttgggatggaaactgcttatataagcttgtaaagctttggttcagaaagcaagagaatgtgtatggaacgttagcaattgctgtcatgcaaatgctgcattttttgcatgtaaaggtacctctttacctccatggtccttaaactaactttttcaagaaacatggtcatttcccacccttcctttacttccagctccccattcatacccaacagtggctaactgaaaggtttgcaaatcctgaaggctttggggcttcttttaaaaagaagrgactgagttgtaaatgtcataagactgagatggttgtaaatgtcccaaccatactgtgtgacatttgctaggaattgttgtaaaatcactggagtgagcgcttgtcctgaggcactgcctctccgcgtgaatgtcactgagtcgcccggttgacccgttgcatgttatgcagcacttgtgacattgttttatctctctagccggtgactcataattaaggaagtcaaaactgtgcaattaacacaaaacctcctcatttgccctcttcacctctccccaacccccgcccccagtcaggaa

SEQ ID NO: 19 - DNA, 727 bp, Homo sapiens
ttagagtttttgaaaaatatttgatttttttaaattgacaaataattgtccatattcatgggatacatagtgatgttttgatacatattaatgtcattatggtgatcacatcaggttaattagcatctccatcctgtcaagcatttatcatttctttgggttggaagcattcaatatcctccttctatttgaaactatgtaatatatgaatgttactatgtatgttactatataagcattcaatattctcctatttgaaactatgtaatatatgattatacatagtaacaatcatatattacataataattgtaaactatgtaataataatattgtaatatatgattgctagatatttgaagctatataatatattattgttaagtataatcatcttacagtggtttaggacactagaactgatttttcctctttagctgtaattttgagacctttaacaaatttctccccatccctctgttccctttacacttttcagcytctagtatcttctattctgtttttacaaagttagagttttggggctgataaatgtttttacctaaatactcatttttgtttccaccaaatggaacatcatgttgtcgtagaatgaaggaatcctttctgtcctatgggttattttcttctccctcaaactgacaatgcaaattaatttgattccccaaagtagtttggaaacagagaagactctaagtgttggcta

SEQ ID NO: 20 - DNA, 601 bp, Homo sapiens
agccaatgggaggaaacctaatttcctgccacaggaaattgttctgctagggaagaatatatttaaggattaaaaccttctgtcagaggaacttgagggagttgctagagaaagacctttgggggtctctcatcaggaagccagtgggtgtgggagagatctcagtcataaatggggaggaggcccaacggcaggttcctgagactgtactcctaatgctagcagctggtcttgggctgggaggaagggctgggccagggagaagagtgggtcataaatagaccattttctgcaaccaagyatatttatggtaacatcagctcagaattaagacttatttattggatttacttaatgggcccagtgccctataaggcctgggagagtgtgttctggacacatcagctatattaaatccagcagctgaaactcccaattcactcttattactttggtaaggattgctacatattttgagtggggcagagggagaaagaaagaacaatgccagggctctgggcagcccagactccgaaccaagaggtgttgttgctgatgcgtaagaagtgaatgagatgtcttccctgtcgtgagtttggga

SEQ ID NO: 21 - DNA, 601 bp, Homo sapiens
cattgaacttgcaacctaactgctgagtgaggacacgtcccttagagacaacaaataataacacctggtctgcacagaagaagatagtagggaccaaagggtgggtctgagcctgcagatcttcagaggacagaaaggggtgaggcggaggcaagtctggacgcctccctggagggggtggggcttaaccctcaccttgaaggaccaggataagttagcctggcagaggctctaggagtacacatttcagacttgggagttgatccggtcctgcgtgagcaaaggcagttatgatccaagygggagtcagaggtggacaggggcgaggacaacagaaccaagagctggaacctctactccaagacctggagctcctacagccacggatgctagcatcatggtgtgcatgtgtgatgtctttgcaaaaggtgtctcagagccaagctgtgaaggccttttaacagaccaccttccttctgattcccagagaccccaccccctggctacctgagtgaagatggagaaaccagtgaccaccagatgaaccacagcatggacgcaggtcagtcatgcagggtcatgctcttattcttaactgatt

SEQ ID NO: 22 - DNA, 715 bp, Homo sapiens
gggcgggtagacgtgagtgaatgaaaattcccccaaactttatgcccaggagaacgagggtctgtggtcagctaagcatacgtgtgcagtttggtgcaaagccacggacagccatgcagactccagccccctgacatttatagacctgtttcagatatacttatttgagtgtttcatcttataattgtgtaatttttcttaaaatagcaacatayacggaatttgaaaataaagtcaaagtgcccataatcccaccaccctaacataactgtctatgtgtgattacagtcctgatgtttgtgcatcaggcatatatatctctgtgccttgccactgagatcatagtgcatatctggtgttttcattctcttttctccttatgttattttgtaaacacgtttccgtatttaggtgtcttgtttatcgttgtgtgttatctcatgatgttttggagtggagggtccattgtttccgcaacatccctccatggttagactgctgggtcagtttttccaagactcaattttctaacccagtagggtccttcagttctccagcttgggataggctttggcatggaacctgccaaagggcttcaaacaaatgaaatatccatttagtttttattgaggggagatttttatattctggtctggtagttttagaaaatgtaagttggattttgtttggaactttattgctgaggcttaatttg

SEQ ID NO: 23 - DNA, 501 bp, Homo sapiens
caaagggcttcaaacaaatgaaatatccatttagtttttattgaggggagatttttatattctggtctggtagttttagaaaatgtaagttggattttgtttggaactttattgctgaggcttaatttgtggctcaggaaaagaaatagttaatatttcttggcgaatcaaagtggattggactggtttttgtgcccatttgcttccagaccttgactgacagcgtcagatgaagaaactcatcatttggratattaggagatgcttgaaaccctttagaacaggctgacctgggcagtgtgggatgtgactttgggagatctgttttaacatttttgtaaaataatatcaccatgagtgccgggctttgcagagggaaggtcagattcctgatatgaaaggactgagttattaaaaacaaactgaaggctagaagcgaagttaacacttgagaaattaaaggccatgatggtgaacttgactccgggctttcagcttttg

(19) United States
(12) Patent Application Publication
(10) Pub. No.: US 2012/0041082 A1
(43) Pub. Date: Feb. 16, 2012

(54) METHODS OF USING SMAD3 AND JAK2 GENETIC VARIANTS TO DIAGNOSE AND PREDICT INFLAMMATORY BOWEL DISEASE

(75) Inventors: Jerome I. Rotter, Los Angeles, CA (US); Kent D. Taylor, Ventura, CA (US); Stephan R. Targan, Santa Monica, CA (US)

(73) Assignee: CEDARS-SINAI MEDICAL CENTER, Los Angeles, CA (US)

(21) Appl. No.: 13/144,376

(22) PCT Filed: Jan. 13, 2010
(86) PCT No.: PCT/US10/20921

§ 371 Date: Jul. 22, 2011

Related U.S. Application Data
(60) Provisional application No. 61/144,290, filed on Jan. 13, 2009.

(51) Int. Cl. C12Q 1/68; A61P 1/00; C07G 99/00

(52) U.S. Cl. 514/789; 435/6.11

(57) ABSTRACT

Disclosed are methods of diagnosing Inflammatory Bowel Disease by determining the presence or absence of genetic variants at SMAD3 and/or JAK2 loci. Provided is a method of diagnosing a Crohn's Disease subtype in an individual by determining the presence or absence of a risk variant at the SMAD3 and/or JAK2 loci.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Crohn's disease (CD) and ulcerative colitis (UC), the two common forms of idiopathic inflammatory bowel disease (IBD), are chronic, relapsing inflammatory disorders of the gastrointestinal tract. Each has a peak age of onset in the second to fourth decades of life and prevalences in European ancestry populations that average approximately 100-150 per 100,000 (D. K. Podolsky, N Engl J Med 347, 417 (2002); E. V. Loftus, Jr., Gastroenterology 126, 1504 (2004)). Although the precise etiology of IBD remains to be elucidated, a widely accepted hypothesis is that ubiquitous, commensal intestinal bacteria trigger an inappropriate, overactive, and ongoing mucosal immune response that mediates intestinal tissue damage in genetically susceptible individuals (D. K. Podolsky, N Engl J Med 347, 417 (2002)). Genetic factors play an important role in IBD pathogenesis, as evidenced by the increased rates of IBD in Ashkenazi Jews, familial aggregation of IBD, and increased concordance for IBD in monozygotic compared to dizygotic twin pairs (S. Vermeire, P. Rutgeerts, Genes Immun 6, 637 (2005)). Moreover, genetic analyses have linked IBD to specific genetic variants, especially CARD15 variants on chromosome 16q12 and the IBD5 haplotype (spanning the organic cation transporters, SLC22A4 and SLC22A5, and other genes) on chromosome 5q31 (S. Vermeire, P. Rutgeerts, Genes Immun 6, 637 (2005); J. P. Hugot et al., Nature 411, 599 (2001); Y. Ogura et al., Nature 411, 603 (2001); J. D. Rioux et al., Nat Genet 29, 223 (2001); V. D. Peltekova et al., Nat Genet 36, 471 (2004)). CD and UC are thought to be related disorders that share some genetic susceptibility loci but differ at others.

The replicated associations between CD and variants in CARD15 and the IBD5 haplotype do not fully explain the genetic risk for CD. Thus, there is need in the art to determine other genes, allelic variants and/or haplotypes that may assist in explaining the genetic risk, diagnosing, and/or predicting susceptibility for or protection against inflammatory bowel disease including but not limited to CD and/or UC.

### SUMMARY OF THE INVENTION

Various embodiments include a method of diagnosing susceptibility to Inflammatory Bowel Disease (IBD) in an individual, comprising obtaining a sample from the individual, assaying the sample to determine the presence or absence of a risk haplotype at the janus kinase 2 (JAK2) genetic locus and/or SMAD family member 3 (SMAD3) genetic locus, and diagnosing susceptibility to IBD in the individual based on the presence of a risk haplotype at the JAK2 genetic locus and/or SMAD3 genetic locus. In another embodiment, the IBD comprises Crohn's disease. In another embodiment, the risk haplotype at the JAK2 genetic locus comprises JAK2 Block 1 Haplotype 1, JAK2 Block 2 Haplotype 1, and/or JAK2 Block 3 Haplotype 3. In another embodiment, the risk haplotype at the JAK2 genetic locus comprises SEQ. ID. NO.: 1, SEQ. ID. NO.: 2, SEQ. ID. NO.: 3, SEQ. ID. NO.: 4, SEQ. ID. NO.: 5, SEQ. ID. NO.: 6 and/or SEQ. ID. NO.: 7. In another embodiment, the risk haplotype at the SMAD3 genetic locus comprises SMAD3 Block 2 Haplotype 4, SMAD3 Block 5 Haplotype 1 and/or SMAD3 Block 6 Haplotype 1. In another embodiment, the risk haplotype at the SMAD3 genetic locus comprises SEQ. ID. NO.: 8, SEQ. ID. NO.: 9, SEQ. ID. NO.: 10, SEQ. ID. NO.: 11, SEQ. ID. NO.: 12, SEQ. ID. NO.: 13 and/or SEQ. ID. NO.: 14. In another embodiment, the risk haplotype at the SMAD3 genetic locus comprises SEQ. ID. NO.: 15 and/or SEQ. ID. NO.: 16. In another embodiment, the risk haplotype at the SMAD3 genetic locus comprises SEQ. ID. NO.: 17, SEQ. ID. NO.: 18, SEQ. ID. NO.: 19, SEQ. ID. NO.: 20, SEQ. ID. NO.: 21, SEQ. ID. NO.: 22 and/or SEQ. ID. NO.: 23.

Other embodiments include a method of determining a low probability of developing Crohn's disease in an individual, relative to a healthy subject, comprising obtaining a sample from the individual, assaying the sample to determine the presence or absence of a protective haplotype at the janus kinase 2 (JAK2) genetic locus and/or SMAD family member 3 (SMAD3) genetic locus, and diagnosing a low probability of developing Crohn's disease in the individual, relative to a healthy subject, based upon the presence of the protective haplotype at the JAK2 and/or SMAD3 genetic locus. In another embodiment, the protective haplotype at the JAK2 genetic locus comprises JAK2 Block 1 Haplotype 3, JAK2 Block 2 Haplotype 2, and/or JAK2 Block 3 Haplotype 1. In another embodiment, the protective haplotype at the SMAD3 genetic locus comprises SMAD3 Block 4 Haplotype 1, SMAD3 Block 5 Haplotype 2, and/or SMAD3 Block 6 Haplotype 2.

Other embodiments include a method of diagnosing a Crohn's disease subtype in an individual, comprising determining the presence of one or more risk variants at the Janus kinase 2 (JAK2) genetic locus and/or SMAD family member 3 (SMAD3) genetic locus, and diagnosing the Crohn's disease subtype in the individual based upon the presence of the one or more risk variants at the JAK2 and/or SMAD3 genetic locus. In another embodiment, the one or more risk haplotypes at the JAK2 genetic locus comprises SEQ. ID. NO.: 1. In another embodiment, the one or more risk variants at the JAK2 genetic locus comprises JAK2 Block 1 Haplotype 1, JAK2 Block 2 Haplotype 1, and/or JAK2 Block 3 Haplotype 3. In another embodiment, the one or more risk variants at the SMAD3 genetic locus comprises SMAD3 Block 2 Haplotype 4, SMAD3 Block 5 Haplotype 1, and/or SMAD3 Block 6 Haplotype 1.

Other embodiments include a method of treating Crohn's disease in an individual, comprising determining the presence of a risk variant at the Janus kinase 2 (JAK2) genetic locus and/or SMAD family member 3 (SMAD3) genetic locus, and treating the individual based upon the presence of the risk variant at the JAK2 genetic locus and/or SMAD3 genetic locus.

Various embodiments include a method of determining the prognosis of Crohn's disease in an individual, comprising determining the presence or absence of one or more risk variants at the janus kinase 2 (JAK2) genetic locus and/or SMAD family member 3 (SMAD3) genetic locus, and prognosing a complicated case of Crohn's disease if the individual demonstrates the presence of one or more risk variants at the JAK3 genetic locus and/or SMAD3 genetic locus. In another embodiment, the one or more risk variants at the JAK2 genetic locus comprises JAK2 Block 1 Haplotype 1, JAK2 Block 2 Haplotype 1, and/or JAK2 Block 3 Haplotype 3. In another embodiment, the one or more risk variants at the SMAD3 genetic locus comprises SMAD3 Block 2 Haplotype 4, SMAD3 Block 5 Haplotype 1, and/or SMAD3 Block 6 Haplotype 1.

Other embodiments include a method of treating Crohn's Disease in an individual, comprising determining the presence of a risk variant at the janus kinase 2 (JAK2) genetic locus in the individual, and treating the individual by inhibiting the JAK2 signaling pathway. In another embodiment, the risk variant at the JAK2 genetic locus comprises SEQ. ID. NO.: 1.

Other features and advantages of the invention will become apparent from the following detailed description, taken in conjunction with the accompanying drawings, which illustrate, by way of example, various embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts, in accordance with an embodiment described herein, a haplotype map and structure of SMAD3, including SMAD3 Blocks 1-3 and corresponding SNPs.

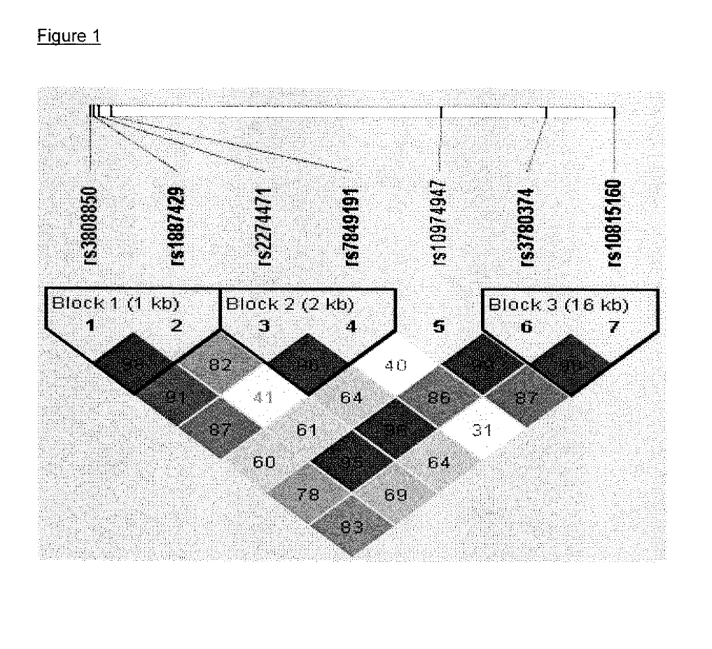

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed, J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

“SMAD3” as used herein refers to SMAD family member 3.

“JAK2” as used herein refers to Janus kinase 2 (a protein tyrosine kinase).

“Haplotype” as used herein refers to a set of single nucleotide polymorphisms (SNPs) on a gene or chromatid that are statistically associated.

“GWAS” as used herein means Genome-Wide Association Study.

“Risk” as used herein refers to an increase in susceptibility to IBD, including but not limited to CD and UC.

“Protective” and “protection” as used herein refer to a decrease in susceptibility to IBD, including but not limited to CD and UC.

“CD” and “UC” as used herein refer to Crohn's disease and ulcerative colitis, respectively.

“F_A” as used herein means frequency in CD.

“F_U” as used herein means frequency in controls.

“P” as used herein means the P value for that association.

“PAR” as used herein refers to population attributable risk, including an estimation of the proportion of cases in the population attributable to the given risk factor.

As used herein, “treatment” or “treating” should be understood to include any indicia of success in the treatment, alleviation or amelioration of an injury, pathology or condition. This may include but not limited to parameters such as abatement, remission, diminishing of symptoms, slowing in the rate of degeneration or decline, making the final point of degeneration less debilitating; improving a patient's physical or mental well-being, or preventing the onset of disease, such as Crohn's disease.

As used herein, “diagnose” or “diagnosis” refers to determining the nature or the identity of a condition or disease. A diagnosis may be accompanied by a determination as to the severity of the disease.

As used herein, “prognostic” or “prognosis” refers to predicting the outcome or prognosis of a disease.

As used herein, the term “biological sample” means any biological material from which nucleic acid molecules can be prepared. As non-limiting examples, the term material encompasses whole blood, plasma, saliva, cheek swab, or other bodily fluid or tissue that contains nucleic acid.

The inventors performed a genome-wide association study testing autosomal single nucleotide polymorphisms (SNPs) on the Illumina HumanHap300 Genotyping BeadChip. Based on these studies, the inventors found single nucleotide polymorphisms (SNPs) and haplotypes that are associated with increased or decreased risk for inflammatory bowel disease, including but not limited to CD. These SNPs and haplotypes are suitable for genetic testing to identify at risk individuals and those with increased risk for complications associated with serum expression of Anti-Saccharomyces cerevisiae antibody, and antibodies to 12, OmpC, and Cbir. The detection of protective and risk SNPs and/or haplotypes may be used to identify at risk individuals predict disease course and suggest the right therapy for individual patients. Additionally, the inventors have found both protective and risk allelic variants for Crohn's Disease and Ulcerative Colitis.

Based on these findings, embodiments of the present invention provide for methods of diagnosing and/or predicting susceptibility for or protection against inflammatory bowel disease including but not limited to Crohn's Disease and ulcerative colitis. Other embodiments provide for methods of prognosing inflammatory bowel disease including but not limited to Crohn's Disease and ulcerative colitis. Other embodiments provide for methods of treating inflammatory bowel disease including but not limited to Crohn's Disease and ulcerative colitis.

The methods may include the steps of obtaining a biological sample containing nucleic acid from the individual and determining the presence or absence of a SNP and/or a haplotype in the biological sample. The methods may further include correlating the presence or absence of the SNP and/or the haplotype to a genetic risk, a susceptibility for inflammatory bowel disease including but not limited to Crohn's Disease and ulcerative colitis, as described herein. The methods may also further include recording whether a genetic risk, susceptibility for inflammatory bowel disease including but not limited to Crohn's Disease and ulcerative colitis exists in the individual. The methods may also further include a prognosis of inflammatory bowel disease based upon the presence or absence of the SNP and/or haplotype. The methods may also further include a treatment of inflammatory bowel disease based upon the presence or absence of the SNP and/or haplotype.

In one embodiment, a method of the invention is practiced with whole blood, which can be obtained readily by non-invasive means and used to prepare genomic DNA, for example, for enzymatic amplification or automated sequencing. In another embodiment, a method of the invention is practiced with tissue obtained from an individual such as tissue obtained during surgery or biopsy procedures.

As disclosed herein, the inventors constructed haplotypes for both the SMAD3 and JAK2 genetic loci and tested for associations in Crohn's Disease subjects. As described in Tables 1-6 herein, various haplotypes and variants were found to have statistically significant associations with Crohn's Disease.

In one embodiment, the present invention provides a method of diagnosing susceptibility to Inflammatory Bowel Disease (IBD) in an individual by determining the presence or absence of a risk variant at the SMAD3 and/or JAK2 genetic locus, where the presence of the risk variant at the SMAD3 and/or JAK2 genetic locus is indicative of susceptibility to IBD in the individual.

In one embodiment, the present invention provides a method of diagnosing a Crohn's Disease (CD) subtype in an individual by determining the presence or absence of a risk variant at the SMAD3 and/or JAK2 genetic locus, where the presence of the risk variant at the SMAD3 and/or JAK2 genetic locus is indicative of the CD subtype in the individual.

In one embodiment, the present invention provides a method of treating CD in an individual by determining the presence of one or more risk variants at the SMAD3 and/or JAK2 genetic locus, and treating the individual.

A variety of methods can be used to determine the presence or absence of a variant allele or haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

A TaqmanB allelic discrimination assay available from Applied Biosystems may be useful for determining the presence or absence of a variant allele. In a TaqmanB allelic discrimination assay, a specific, fluorescent, dye-labeled probe for each allele is constructed. The probes contain different fluorescent reporter dyes such as FAM and VICTM to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., “3′-minor groove binder-DNA probes increase sequence specificity at PCR extension temperature, “Nucleic Acids Research 28:655-661 (2000)). Minor grove binders include, but are not limited to, compounds such as dihydrocyclopyrroloindole tripeptide (DPI).

Sequence analysis also may also be useful for determining the presence or absence of a variant allele or haplotype.

Restriction fragment length polymorphism (RFLP) analysis may also be useful for determining the presence or absence of a particular allele (Jarcho et al. in Dracopoli et al., Current Protocols in Human Genetics pages 2.7.1-2.7.5, John Wiley & Sons, New York; Innis et al., (Ed.), PCR. Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well known assay that may be used to detect a SNP or a haplotype. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261 (1993); White et al., Genomics 12:301-306 (1992)).

The technique of single strand conformational, polymorphism (SSCP) also may be used to detect the presence or absence of a SNP and/or a haplotype (see Hayashi, K., Methods Applic. 1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a SNP and/or a haplotype are known in the art and useful in the methods of the invention. Other well-known approaches for determining the presence or absence of a SNP and/or a haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or haplotype(s) is to be determined, individual alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the present invention for diagnosing or predicting susceptibility to or protection against CD in an individual may be practiced using one or any combination of the well known assays described above or another art-recognized genetic assay.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

TABLE 1
Significant JAK2 haplotypes and variants
Table 1.
Table 1 describes various JAK2 haplotypes with statistically significant associations.
The “B” corresponds with the Block number, and the “H” corresponds with the Haplotype
number.

| JAK2 | | Case | Control | P | OR | PAR |
|---|---|---|---|---|---|---|
| best SNP from GWAS(meta- | [SEQ. ID. NO.: 1] | 0.374 | 0.348 | 3.46 x 10⁻⁹ | 1.1 | 3.40% |
| analysis | rs10758669 allele C | | | | | |
| haplotypes of JAK2 from Cedars | B1H1carrier | 0.683 | 0.640 | 1.8 x 10⁻² | 1.2 | 11.30% |
| | B1H3carrier | 0.466 | 0.509 | 2.7 x 10⁻² | 0.8 | −10.20% |
| | B2H1carrier | 0.630 | 0.591 | 4 x 10⁻² | 1.2 | 10.60% |
| | B2H2homozygotes | 0.115 | 0.150 | 6.2 x 10⁻³ | 0.7 | −4.50% |
| | B3H1carrier | 0.664 | 0.710 | 1.1 x 10⁻² | 0.8 | −14.20% |
| | B3H3carrier | 0.496 | 0.451 | 2 x 10⁻² | 1.2 | 8.30% |

### Example 2

TABLE 2
Haplotype information of JAK2
Table 2.
The JAK2 haplotypes referenced in Table 1 above and herein are defined in Table 2 below,
where JAK2 Blocks 1-3 and haplotypes 1-3 are defined by listed SNPs and the corresponding
allele. The “B” corresponds with the Block number, and “H” corresponds with the Haplotype
number.

| | | Haplotype 1 | Haplotype 2 | Haplotype 3 |
|---|---|---|---|---|
| Block | SNPs | Alleles | Alleles | Alleles |
| 1 | Rs3808850 | T | T | A |
| | [SEQ. ID. NO.: | | | |
| | 2] | | | |
| | Rs1887429 | C | A | C |
| | [SEQ. ID. NO.: | | | |
| | 3] | | | |
| 2 | Rs2274471 | A | A | G |
| | [SEQ. ID. NO.: | | | |
| | 4] | | | |
| | Rs7849191 | G | A | G |
| | [SEQ. ID. NO.: | | | |
| | 5] | | | |
| 3 | Rs3780374 | G | G | A |
| | [SEQ. ID. NO.: | | | |
| | 6] | | | |
| | Rs10815160 | A | C | A |
| | [SEQ. ID. NO.: | | | |
| | 7] | | | |

### B1H1:TC 0.42

### B1H2:TA 0.3

### 1H3:AC 0.28

### B2H1:AG 0.38

### B2H2:AA 0.36

### B2H3:GG 0.26

### B3H1:GA 0.44

### B3H2:GC 0.28

### B3H3:AA 0.28

### Example 3

TABLE 3
Table 3 - Significant SMAD3 haplotypes and variants
Table 3 describes various SMAD3 haplotypes with statistically
significant associations. The “B” corresponds with the
Block number, and the “H” corresponds with the Haplotype number.

| SMAD3 | | Case | Control | P | OR | PAR |
|---|---|---|---|---|---|---|
| best SNP | not identified | | | | | |
| from GWAS | | | | | | |
| (meta- | | | | | | |
| analysis) | | | | | | |
| haplotypes | B2H4carrier | 0.129 | 0.100 | 2.3 × 10⁻² | 1.3 | 3% |
| of SMAD3 | | | | | | |
| from Cedars | | | | | | |
| | B4H1carrier | 0.753 | 0.793 | 1.5 × 10⁻² | 0.8 | −15.90% |
| | B5H1carrier | 0.736 | 0.691 | 1.1 × 10⁻² | 1.2 | 12.10% |
| | B5H2carrier | 0.476 | 0.531 | 4.3 × 10⁻³ | 0.8 | −10.60% |
| | B6H1carrier | 0.504 | 0.452 | 8.2 × 10⁻³ | 1.2 | 8.30% |
| | B6H2homo | 0.051 | 0.082 | 1.2 × 10⁻³ | 0.6 | −3.20% |

### Example 4

### Table 4(a)-4(d)—Haplotype information of SMAD3

TABLE 4(a)
Table 4(a) describes haplotype information on
Block 2 of SMAD3, specifically for SMAD3 Block 2
Haplotype 4. The “B” corresponds with the
Block number, and “H” corresponds with
the Haplotype number.

| SNPs | Alleles |
|---|---|
| Rs9972423 [SEQ. ID. NO.: 8] | T |
| Rs2118611 [SEQ. ID. NO.: 9] | G |
| Rs11071933 [SEQ. ID. NO.: 10] | C |
| Rs1438386 [SEQ. ID. NO.: 11] | G |
| Rs718663 [SEQ. ID. NO.: 12] | G |
| Rs7163381 [SEQ. ID. NO.: 13] | A |
| Rs920293 [SEQ. ID. NO.: 14] | A |

B2H1: AAGAAGA 0.37
B2H2: TAGAAGA 0.22
B2H3: TGCGAAA 0.11
B2H4: TGCGGAA 0.06
B2H5: TACGAAG 0.06
B2H6: TAGGAGA 0.06

TABLE 4(b)
Table 4(b) describes haplotype information
on Block 4 of SMAD3, specifically SMAD3
Block 4 Haplotype I. The “B” corresponds
with the Block number, and “H” corresponds
with the Haplotype number.

| SNPs | Alleles |
|---|---|
| Rs745103 | A |
| Rs12439792 | A |
| Rs17293443 | A |

B4H1:AAA 0.52
B4H2:GAG 0.21
B4H3:GAA 0.15
B4H4:GTA 0.12

TABLE 4(c)
Table 4(c) describes haplotype information
on Block 5 of SMAD3, specifically SMAD3
Block 5 Haplotype 1 and 2. The “B”
corresponds with the Block number, and
“H” corresponds with the Haplotype number.

| | Haplotype 1 | Haplotype 2 |
|---|---|---|
| SNPs | Alleles | Alleles |
| Rs893473 | G | G |
| [SEQ. ID. NO.: 15] | | |
| Rs2289263 | C | A |
| [SEQ. ID. NO.: 16] | | |

B5H1:GC 0.48
B5H2:GA 0.29
B5H3:AA 0.23

TABLE 4(d)
Table 4(d) describes haplotype information
on Block 6 of SMAD3, specifically SMAD3
Block 6 Haplotype 1 and 2. The “B”
corresponds with the Block number, and
“H” corresponds with the Haplotype number.

| | Haplotype 1 | Haplotype 2 |
|---|---|---|
| SNPs | Alleles | Alleles |
| Rs2033785 | G | G |
| [SEQ. ID. NO.: 17] | | |
| Rs11637659 | G | G |
| [SEQ. ID. NO.: 18] | | |
| Rs10152307 | G | A |
| [SEQ. ID. NO.: 19] | | |
| Rs4776900 | G | G |
| [SEQ. ID. NO.: 20] | | |
| Rs7179840 | A | G |
| [SEQ. ID. NO.: 21] | | |
| Rsl1071939 | A | A |
| [SEQ. ID. NO.: 22] | | |
| Rs16950687 | G | A |
| [SEQ. ID. NO.: 23] | | |

B6H1:GGGGAAG 0.29
B6H2:GGAGGAA 0.24
B6H3:CGGGAAA 0.24
B6H4:GAGAGAA 0.11
B6H5:GAGGAGA 0.07

### Example 5

TABLE 5
Table 5 - Additional associations with JAK2 Haplotypes
Table 5 describes information on additional JAK2 haplotype association.

| Locus | Hap | F_A | F_U | CHISQ | DF | P |
|---|---|---|---|---|---|---|
| JAK2 | CTC | 0.3959 | 0.364 | 5.885 | 1 | 0.01527 |

SNPs that define the alternative JAK2 haplotype: rs10758669|rs3808850|rs1887429
rs10758669: C is the associated allele, other allele is A
rs3808850: T is the associated allele, other allele is T
rs1887429: C is the associated allele, other allele is A

### Example 5

TABLE 6
Table 6 - Additional associations with SMAD3 Haplotypes
Table 6 describes information on an
additional SMAD3 haplotype association.

| Locus | Hap | F_A | F_U | CHISQ | DF | P |
|---|---|---|---|---|---|---|
| SMAD3 | GC | 0.2391 | 0.2061 | 8.563 | 1 | 0.003432 |

SNPS that define the alternative SMAD3 haplotype: rs2118611|rs11071933
rs2118611: G is the associated allele, other allele is A
rs11071933: C is the associated allele, other allele is G

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive.

Various embodiments of the invention are described above in the Description of the Invention. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

1. A method of diagnosing susceptibility to Inflammatory Bowel Disease (IBD) in an individual, comprising:
obtaining a sample from the individual;
assaying the sample to determine the presence or absence of a risk haplotype at the janus kinase 2 (JAK2) genetic locus and/or SMAD family member 3 (SMAD3) genetic locus; and
diagnosing susceptibility to IBD in the individual based on the presence of a risk haplotype at the JAK2 genetic locus and/or SMAD3 genetic locus.

2. The method of claim 1, wherein IBD comprises Crohn's disease.

3. The method of claim 1, wherein the risk haplotype at the JAK2 genetic locus comprises JAK2 Block 1 Haplotype 1, JAK2 Block 2 Haplotype 1, and/or JAK2 Block 3 Haplotype 3.

4. The method of claim 1, wherein the risk haplotype at the JAK2 genetic locus comprises SEQ. ID. NO.: 1, SEQ. ID. NO.: 2, SEQ. ID. NO.: 3, SEQ. ID. NO.: 4, SEQ. ID. NO.: 5, SEQ. ID. NO.: 6 and/or SEQ. ID NO.: 7.

5. The method of claim 1, wherein the risk haplotype at the SMAD3 genetic locus comprises SMAD3 Block 2 Haplotype 4, SMAD3 Block 5 Haplotype 1 and/or SMAD3 Block 6 Haplotype 1.

6. The method of claim 1, wherein the risk haplotype at the SMAD3 genetic locus comprises SEQ. ID. NO.: 8, SEQ. ID. NO.: 9, SEQ. ID. NO.: 10, SEQ. ID. NO.: 11, SEQ. ID. NO.: 12, SEQ. ID. NO.: 13 and/or SEQ. ID. NO.: 14.

7. The method of claim 1, wherein the risk haplotype at the SMAD3 genetic locus comprises SEQ. ID. NO.: 15 and/or SEQ. ID. NO.: 16.

8. The method of claim 1, wherein the risk haplotype at the SMAD3 genetic locus comprises SEQ. ID. NO.: 17, SEQ. ID. NO.: 18, SEQ. ID. NO.: 19, SEQ. ID. NO.: 20, SEQ. ID. NO.: 21, SEQ. ID. NO.: 22 and/or SEQ. ID. NO.: 23.

9. A method of determining a low probability of developing Crohn's disease in an individual, relative to a healthy subject, comprising:
obtaining a sample from the individual;
assaying the sample to determine the presence or absence of a protective haplotype at the janus kinase 2 (JAK2) genetic locus and/or SMAD family member 3 (SMAD3) genetic locus; and
diagnosing a low probability of developing Crohn's disease in the individual, relative to a healthy subject, based upon the presence of the protective haplotype at the JAK2 and/or SMAD3 genetic locus.

10. The method of claim 9, wherein the protective haplotype at the JAK2 genetic locus comprises JAK2 Block 1 Haplotype 3, JAK2 Block 2 Haplotype 2, and/or JAK2 Block 3 Haplotype 1.

11. The method of claim 9, wherein the protective haplotype at the SMAD3 genetic locus comprises SMAD3 Block 4 Haplotype 1, SMAD3 Block 5 Haplotype 2, and/or SMAD3 Block 6 Haplotype 2.

12. A method of diagnosing a Crohn's disease subtype in an individual, comprising:
determining the presence of one or more risk variants at the janus kinase 2 (JAK2) genetic locus and/or SMAD family member 3 (SMAD3) genetic locus; and
diagnosing the Crohn's disease subtype in the individual based upon the presence of the one or more risk variants at the JAK2 and/or SMAD3 genetic locus.

13. The method of claim 12, wherein the one or more risk haplotypes at the JAK2 genetic locus comprises SEQ. ID. NO.: 1.

14. The method of claim 12, wherein the one or more risk variants at the JAK2 genetic locus comprises JAK2 Block 1 Haplotype 1, JAK2 Block 2 Haplotype 1, and/or JAK2 Block 3 Haplotype 3.

15. The method of claim 12, wherein the one or more risk variants at the SMAD3 genetic locus comprises SMAD3 Block 2 Haplotype 4, SMAD3 Block 5 Haplotype 1, and/or SMAD3 Block 6 Haplotype 1.

16. A method of treating Crohn's disease in an individual, comprising:
determining the presence of a risk variant at the janus kinase 2 (JAK2) genetic locus and/or SMAD family member 3 (SMAD3) genetic locus; and
treating the individual based upon the presence of the risk variant at the JAK2 genetic locus and/or SMAD3 genetic locus.

17. A method of determining the prognosis of Crohn's disease in an individual, comprising:
determining the presence or absence of one or more risk variants at the janus kinase 2 (JAK2) genetic locus and/or SMAD family member 3 (SMAD3) genetic locus; and
prognosing a complicated case of Crohn's disease if the individual demonstrates the presence of one or more risk variants at the JAK3 genetic locus and/or SMAD3 genetic locus.

18. The method of claim 17, wherein the one or more risk variants at the JAK2 genetic locus comprises JAK2 Block 1 Haplotype 1, JAK2 Block 2 Haplotype 1, and/or JAK2 Block 3 Haplotype 3.

19. The method of claim 17, wherein the one or more risk variants at the SMAD3 genetic locus comprises SMAD3 Block 2 Haplotype 4, SMAD3 Block 5 Haplotype 1, and/or SMAD3 Block 6 Haplotype 1.

20. A method of treating Crohn's Disease in an individual, comprising:
determining the presence of a risk variant at the janus kinase 2 (JAK2) genetic locus in the individual; and
treating the individual by inhibiting the JAK2 signaling pathway.

21. The method of claim 20, wherein the risk variant at the JAK2 genetic locus comprises SEQ. ID. NO.: 1.
